(19) United States
(12) Patent Application Publication
(10) Pub. No.: US 2023/0357315 A1
(43) Pub. Date: Nov. 9, 2023

(54) METHOD OF PURIFYING AN Fc-FUSION PROTEIN

(71) Applicant: DR. REDDY'S LABORATORIES LIMITED, Hyderabad (IN)

(72) Inventors: Ravichandran RAMAKRISHNAN, Saligramam Chennai (IN); Gopinath GOVINDARAJAN, Mylapore Chennai (IN); Krishna Prasad CHELLAPILLA, Vanasthalipuram Hyderabad (IN)

(21) Appl. No.: 17/916,174

(22) PCT Filed: Mar. 10, 2021
(86) PCT No.: PCT/IN2021/050236

§ 371 Date: Sep. 30, 2022

(30) Foreign Application Priority Data
Mar. 11, 2020 (IN) 202041010286

(51) Int. Cl. C07K 1/36; C07K 16/06

(52) U.S. Cl. CPC C07K 1/36; C07K 16/065; C07K 2319/30

(57) ABSTRACT

The present invention discloses a method for the purification of Fc-fusion proteins from the contaminants. In particular, the disclosed method describes a process for the purification of Fc-fusion proteins using a specific order of chromatographic steps. The specific order of chromatography steps as disclosed results in reducing contaminants such as high-molecular weight aggregates and sialylated isoforms and obtaining a purified Fc-fusion protein composition.

### FIELD OF INVENTION

The present invention relates to protein purification methods. In particular, the invention discloses a method of purifying fusion proteins using a particular order of chromatographic steps.

### BACKGROUND

Fc-fusion proteins are bioengineered polypeptides that join the crystallizable fragment (Fc) domain of an antibody with another biologically active protein domain to generate a molecule with unique structure-function properties and significant therapeutic potential. The gamma immunoglobulin (IgG) isotype is often used as the basis for generating Fc-fusion proteins because of favorable characteristics such as recruitment of effector function and increased plasma half-life. Given the range of proteins that can be used as fusion partners, Fc-fusion proteins have numerous biological and pharmaceutical applications, which has launched Fc-fusion proteins into the forefront of drug development.

Fc-fusion proteins can be commercially manufactured using platform upstream and downstream methods based for monoclonal antibodies (mAb). However, Fc-fusion proteins, receptor domains generally contain one or more glycosylation sites (both N- and O-linked) in contrast to single glycosylation site for mAbs. Also, the oligosaccharide structures are more varied and complex (complex and high mannose; bi-, tri- and tetra-antennary) in their receptor domains than IgG Fc (complex, bi-anntennary) and can contain more sialic acid residues. The latter can shift the isoelectric point (pI) of Fc-fusion proteins into an acidic pH range and impart significantly more charge heterogeneity on them than that of the conventional mAbs. Hence, there are unique attributes of Fc-Fusion proteins that would require optimization or redesigning of the commercial process used for the manufacture of mAbs.

Therapeutic Fc-fusion proteins, including CTLA4-Ig fusion proteins (for example, abatacept), are produced by recombinant DNA technology and such proteins expressed by recombinant DNA technology are typically associated with impurities such as host cell proteins (HCP), host cell DNA (HCD), monomers, high molecular weight (HMW) aggregates, sialylated isoforms, viruses, endotoxins etc. The presence of these impurities is a potential health risk, and hence their removal from the final product is a regulatory requirement and poses significant challenge in the development of methods for the purification of therapeutic proteins, in general and CTLA4-Ig fusion protein in particular.

Although, chromatography is a widely used technique in the purification of proteins, nevertheless, choosing a particular chromatographic step or a combination of chromatographic steps for effective removal of impurities without affecting the stability and function of the protein is essential, yet rendering it to be cost-effective is a perpetual process and research in itself.

The principle object of the present invention is to provide a method of chromatographic purification of Fc-fusion proteins for the effective removal of various kinds of impurities while, at the same time, ensuring the economic effectiveness in achieving the objective.

### SUMMARY

The present invention discloses a method involving a combination of one or more chromatographic steps for the purification of Fc-fusion protein. In particular, the invention discloses that a specific order of chromatographic steps results in the purification process of Fc-fusion protein, wherein specific impurities are removed or controlled only when the order of chromatographic steps is followed. In adopting a particular order of chromatographic steps, the invention discloses a method wherein multiple interspacing buffer exchange or filtration steps are eliminated thus resulting in a purification method that is technically and economically efficient.

The method disclosed in the present invention effectively removes high-molecular weight (HMW) aggregates, non-covalent dimers and sialylated isoforms (2%-7%), to yield a purified CTLA4-Ig fusion protein composition, without affecting recovery.

The method is used for the purification of therapeutic Fc-fusion proteins. In particular, the method disclosed in the invention is capable of being used at commercial scale for controlling the level of BMW aggregates, monomers, viruses, endotoxins and sialylated isoforms and obtaining a purified composition of the said CTLA4-Ig fusion protein.

### DETAILED DESCRIPTION OF THE INVENTION

### Definitions

“Fc-fusion protein” is a protein that contains an Fc region of an immunoglobulin fused or linked to a polypeptide. The heterologous polypeptide fused to the Fc region may be a polypeptide from a protein other than an immunoglobulin protein. For instance, the heterologous polypeptide may be a ligand polypeptide, a receptor polypeptide, a hormone, cytokine, growth factor, an enzyme, or other polypeptide that is not a component of an immunoglobulin. Such Fc-fusion proteins may comprise an Fc region fused to a receptor or fragment thereof or a ligand from a receptor including, but not limited to, any one of the following receptors: both forms of TNFR (referred to as p55 and p′75), Interleukin-1 receptors types I and II (as described in EP Patent No. 0460846, U.S. Pat. Nos. 4,968,607, 5,767,064, which are incorporated by reference herein in their entirety), Interleukin-2 receptor, Interleukin-4 receptor (as described in EP Patent No. 0 367 566 and U.S. Pat. No. 5,856,296, which are incorporated by reference herein in their entirety), Interleukin-15 receptor, Interleukin-17 receptor, Interleukin-18 receptor, granulocyte-macrophage colony stimulating factor receptor, granulocyte colony stimulating factor receptor, receptors for oncostatin-M and leukemia inhibitory factor, receptor activator of NF-kappa B (RANK, as described in U.S. Pat. No. 6,271,349, which is incorporated by reference herein in its entirety), VEGF receptors, EGF receptor, FGF receptors, receptors for TRAIL (including TRAIL receptors 1,2,3, and 4), and receptors that comprise death domains, such as Fas or Apoptosis-Inducing Receptor (AIR). Fc fusion proteins also include peptibodies, such as those described in WO 2000/24782, which is hereby incorporated by reference in its entirety.

As used herein, CTLA-4-Ig fusion protein refers to a protein that links the extracellular domain of human cytotoxic T-lymphocyte associated antigen 4 (CTLA-4) to the modified Fc (hinge, CH2 and CH3 domains) region of human immunoglobulin G1. It is a homodimer of two polypeptide chains connected together through one disulfide bond in the CTLA-4 domain.

“High molecular weight aggregates” as referred herein encompasses association of at least two molecules of a protein of interest, e.g., Fc-Fusion protein. The association of at least two molecules of a protein of interest may arise by any means including, but not limited to, non-covalent interactions such as, e.g., charge-charge, hydrophobic and van der Waals interactions; and covalent interactions such as, e.g., disulfide interaction or non-reducible crosslinking. An aggregate can be a dimer, trimer, tetramer, or a multimer greater than a tetramer, etc. Aggregate concentration can be measured in a protein sample using Size Exclusion Chromatography (SEC), a well-known and widely accepted method in the art.

As used herein, the term “sialylation” refers to the addition of sialic acid residues to a protein. Sialic acid is a common name for a family of unique nine-carbon monosaccharides, which can be linked to other oligosaccharides. Two sialic acid family members are N-acetyl neuraminic acid, abbreviated as Neu5Ac or NANA, and N-glycolyl neuraminic acid, abbreviated as Neu5Gc or NGNA. The most common form of sialic acid found in humans is NANA. N-acetylneuraminic acid (NANA) is the primary sialic acid species present in CTLA4-Ig molecules. However, it should be noted that minor but detectable levels of N glycolylneuraminic acid (NGNA) are also present in CTLA4-Ig molecules. Sialic acid is the terminal residue of both N-linked and O-linked oligosaccharides. The level of Sialylation can be estimated in a protein sample using hydrophilic interaction chromatography, a technique well known in the art.

The “composition” to be purified herein comprises the protein of interest and one or more contaminants. The composition may be “partially purified” (i.e., having been subjected to one or more purification steps) or may be obtained directly from a host cell or organism producing the antibody (e.g., the composition may comprise harvested cell culture fluid).

The term “Hydrophobic Interaction Chromatography” refers to a form of chromatography that uses a chromatographic support with functional groups that separate proteins on the basis of their hydrophobicity.

The term “Mixed Mode Chromatography” refers to a form of chromatography that uses a chromatographic support with at least two unique types of functional groups, each interacting with the molecule or protein of interest. Mixed mode chromatography generally uses ligands that have more than one type of interaction with target proteins and/or impurities. For example, a charge-charge type of interaction and/or a hydrophobic or hydrophilic type of interaction, or an electroreceptor-donor type interaction. In general, based on the difference in the total interaction, the target protein and one or more impurities can be separated under various conditions.

The term “Anion Exchange Chromatography” refers to a form of ion-exchange chromatography that uses a support with functional groups that exchanges anions.

The term “bind and elute mode” as used herein refers to a process wherein the target protein binds to the chromatographic support, and is subsequently eluted.

The term “flow-through mode” as used herein refers to a process wherein the target protein is not bound to the chromatographic support but instead obtained in the unbound or “flow-through” fraction during loading or post load wash of the chromatography support.

### DETAILED DESCRIPTION OF THE EMBODIMENTS

The present invention discloses a method comprising a specific order of chromatography steps to achieve the purification of an Fc-fusion protein from the contaminants. Surprisingly, the invention discloses a method wherein the chromatographic steps employed in a specific order (viz., in a specific sequence) alone influences the control and/or removal of impurities in an

Fc-fusion protein composition. When the order is altered, the impurity content either increases or is not controlled in the composition, thus affecting the purity of the protein composition.

In an embodiment, the invention provides a method of purification of an Fc-fusion protein comprising hydrophobic interaction chromatography step and anion exchange chromatography, wherein the hydrophobic interaction chromatographic step always precedes the anion exchange chromatographic step.

In yet another embodiment, the invention provides a method of purification of an Fc-fusion protein comprising a combination of chromatographic steps, wherein the chromatographic steps include an affinity chromatography, followed by a hydrophobic interaction chromatography, followed by an anion exchange chromatography.

In an embodiment, the invention provides a method for the purification of an Fc-Fusion protein comprising chromatography steps of following order;
a) Affinity Chromatography
b) Hydrophobic Interaction chromatography
c) Mixed mode chromatography and
d) Anion-exchange chromatography

In any of the above mentioned embodiments, the anion-exchange chromatography is performed after HIC step to control the sialylated isoforms.

In any of the above mentioned embodiments, the affinity chromatography and/or the anion-exchange chromatography are performed in bind-elute mode.

In any of the above mentioned embodiments, the hydrophobic interaction chromatography and/or mixed mode chromatography are performed in flow-through mode.

In any of the above mentioned embodiments, the affinity chromatography is used as a capture step with significant removal of impurities, host cell protein and host cell DNA.

In any of the above mentioned embodiments, the hydrophobic interaction chromatography is performed to control the high molecular weight (HMW) aggregates content to about 1.5% or lower, and non-covalent dimers content to below detectable limit, in the flow-through fraction (viz., the HIC output) comprising the protein of interest.

In any of the above mentioned embodiments, the mixed mode chromatography is placed after the hydrophobic interaction chromatographic step, but before the anion exchange chromatographic step to reduce the HMW impurities to ≤1.0% and to the control virus and endotoxin levels in obtaining a purified Fc-fusion protein composition. The mixed mode chromatographic step when placed between the HIC and anion exchange steps, eliminates the need for a buffer exchange and additional filtration and concentration steps, in turn preventing the loss of protein.

In an embodiment, the invention provides a method of purification of an Fc-fusion protein comprising a combination of chromatographic steps, wherein the combination includes an anion exchange chromatography, and wherein the anion exchange chromatography is performed as the final chromatographic step to obtain a purified Fc-protein composition.

In any of the above mentioned embodiments, the fusion protein is CTLA4-Ig fusion protein. In particular, the fusion protein is abatacept.

In any of the above mentioned embodiments, the purification method may employ use of one or more steps such as viral inactivation, filtration and diafiltration. These steps may be interspersed between the chromatographic steps or after all the chromatographic steps.

The embodiments mentioned herein may include one or more neutralization steps.

The invention is more fully understood by reference to the following examples. These examples should not, however, be construed as limiting the scope of the invention.

### EXAMPLES

A CTLA4-Ig fusion protein, more specifically abatacept, was cloned and expressed in a Chinese Hamster Ovary cell line and the cell culture broth containing the expressed fusion protein was harvested, clarified and subjected to Protein-A affinity chromatography as described below.

### Example 1

### Protein-A Chromatography

The clarified cell culture broth was loaded onto the Protein-A chromatography column (KANEKA KanCap A™ 3G) that was pre-equilibrated with a buffer (pH 7.0) containing 30 mM Tris acetate and 150 mM sodium chloride. The column was then washed with the same buffer, followed by a high-salt wash with 200 mM Tris Acetate, 1 M NaCl. The column was then washed with 30 mM acetate (pH 6.0). The bound protein was eluted using elution buffer containing 120 mM acetate at a pH of 3.5. The eluate from Protein-A affinity chromatography was subjected to low-pH incubation (pH: 3.5±0.2) and depth filtration.

The low-pH-incubated sample comprising the protein of interest was subjected to further chromatographic steps and experimentation was done to evaluate the process efficiency in terms of impurity removal by varying the placement of the chromatographic steps in different order, which is elucidated in the Table 1, and the methods are exemplified in examples 2, 3 and 4.

TABLE 1
Order of chromatographic steps for the purification of an Fc-fusion protein
Order of chromatographic steps

Affinity→AEX→HIC→MMC
Affinity→AEX→HIC
Affinity→HIC→ AEX→MMC
Affinity→HIC→MMC→AEX

### Example 2

### Hydrophobic Interaction Chromatography (HIC)

The low-pH incubated solution as exemplified in example 1 was subjected to hydrophobic interaction chromatography, operated in flow-through mode. HIC was carried out on a linear butyl support (Capto™ Butyl). Column chromatography conditions are listed in Table 2 and buffer details are captured in Table 3. Fractionation of the HIC flow-through comprising the protein of interest was done based on UV signal, and representative fractions were analyzed by Capillary Electrophoresis—Sodium dodecyl sulphate (CE-SDS) for non-covalent dimer content and size exclusion chromatography—high performance liquid chromatography (SEC-HPLC) for HMW aggregate content. % UMW content and % non-covalent dimer content before and after HIC step are shown in Table 4.

TABLE 2
Chromatography conditions for hydrophobic
interaction chromatography

| Column ID | Hi-scale 50 |
|---|---|
| Resin | Capto ™ Butyl |
| Bed Height (cm) | 21.8 |
| Column Volume (mL) | 428.04 |
| Residence Time (min) | 6.0 |
| (loading and Post-load wash) | |
| Flow Rate (mL/min) | 71.34 |
| Load Factor (g/L) | 30-40 |

TABLE 3
Buffer details for hydrophobic interaction chromatography

| Stage | Buffers used | Target pH |
|---|---|---|
| Equilibration | 20 mM Tris + 0.5M Ammonium | 7.8 |
| (Buffer A) | sulphate | |
| Loading | Neutralized eluate diluted 1:1 (v/v) | 7.5 |
| | with 2X stock of equilibration buffer | |
| PLW | 20 mM Tris + 0.5M Ammonium | 7.5 |
| | sulphate | |
| Regeneration | 0.5M Sodium hydroxide | NA |
| | Purified water | NA |

TABLE 4
% HMW aggregates and % non-covalent
dimer data in HIC and HIC flow-through

| | Batch 1 | Batch 2 | Batch 3 | Batch 1 | Batch 2 | Batch 3 |
|---|---|---|---|---|---|---|
| Sample | HMW (%) | | | Non-covalent dimer (%) | | |
| HIC load | 26.0 | 24.0 | 24.0 | 6.0 | 5.0 | 6.0 |
| HIC flow- | 1.1 | 1.1 | 1.3 | 0.0 | 0.0 | 0.0 |
| through | | | | | | |

### Example 3

### Mixed Mode Chromatography

The flow-through fraction comprising the protein of interest obtained from HIC, as exemplified in example 2, was loaded onto ceramic hydroxyapatite (CHT Type I, 80 micron) support after diafiltration in 10 mM phosphate buffer comprising 15 mM NaCl, pH 7.5. CHT chromatography was operated in flow-through mode. Fractionation of the HIC flow-through comprising the protein of interest was done based on UV signal, and representative fractions were analyzed by size exclusion chromatography—high performance liquid chromatography (SEC-HPLC) for HMW aggregate content. The chromatographic details are listed in Table 5 and the % HMW content before and after the MMC step is shown in Table 6.

TABLE 5
Chromatography conditions for mixed mode chromatography

| | |
|---|---|
| Resin | CHT Type I, 80 micron |
| Load pH | 7.4 ± 0.2 |
| Load conductivity | 3.0 ± 0.2 mS/cm |
| Load factor (g/L of resin) | 25-50 |
| Residence time (min) | 6 |
| Equilibration/post load | 10 mM sodium phosphate + 15 |
| wash buffer | mM Sodium chloride pH 7.5 |

TABLE 6
% HMW aggregates data for MMC load and MMC flow-through

| | | |
|---|---|---|
| Batch 1 | Batch 2 | Batch 3 |

| | |
|---|---|
| Sample | HMW (%) |

| | | | |
|---|---|---|---|
| MMC load | 1.1 | 1.1 | 1.3 |
| MMC flow-though | 0.3 | 0.4 | 0.6 |

### Example 4

### Anion Exchange Chromatography

The flow-through fraction comprising the protein of interest obtained from mixed mode chromatography (MMC), as exemplified in example 3, was loaded onto an anion exchange chromatography (AEX) support (DEAE Sepharose 4FF). AEX was operated in bind-elute mode, wherein the CTLA4-Ig fusion protein was bound to the column and eluted later using an elution buffer solution. Before loading the protein of interest, the chromatographic support was equilibrated with an equilibration buffer solution (pH 7.5±0.3) containing 60 mM Tris-Acetate. The chromatographic support was then washed with the same buffer. The bound CTLA4-Ig fusion protein was eluted using an elution buffer solution containing 60 mM Tris-Acetate, 0.12 M NaCl (pH 7.5, conductivity 14 mS/cm) by a step salt gradient. The chromatographic support was then washed with a wash buffer solution comprising 60 mM Tris-Acetate, 0.5 M NaCl (pH 7.5). The eluate of AEX chromatography was analysed for percentage of di- and tri-sialylated isoforms using a hydrophilic interaction chromatography (HILIC-UPLC) method. The results of sialic acid isoform quantification are shown in Table 7.

TABLE 7
Percentage of Sialic acid isoforms in AEX load and AEX eluate

| | | |
|---|---|---|
| Batch 1 | Batch 2 | Batch 3 |

| | |
|---|---|
| Sample | Di + Tri Sialic acid isoforms (%) |

| | | | |
|---|---|---|---|
| AEX load | 35.3 | 33.8 | 28.4 |
| AEX eluate | 31 | 30.7 | 25.7 |

The eluate from AEX chromatography may then be subjected to one or more ultra/dia filtration steps and buffer exchange steps and/or sterile filtration to obtain a therapeutic composition to be administered for human use.

The method comprising specific order of chromatography steps viz., Affinity>>HIC>>AEX and Affinity→HIC→MMC→AEX, resulted in a purified CTLA4-IgG fusion protein composition. Each chromatography step in the proposed order has significance in controlling the product, process and host cell impurities such as significant reduction in HCP, HCD, UMW aggregates, including sialylated isoforms. Specifically, MMC step before an AEX, in addition to the removal/control of impurities also avoids excess filtration steps thereby reducing production cost and loss in protein yield. in contrast to the below mentioned order of chromatography steps illustrated in the Table 8 that showed significant reduction of UMW aggregates but no significant control on Di+Tri sialic acid isoforms.

TABLE 8
Comparison of order of chromatography steps for
the evaluation of various contaminant levels

| | | % Non- | | % Di + Tri SA |
|---|---|---|---|---|
| Order of chromatographic steps | HCP/HCD | covalent dimer | % HMW | variants |
| Affinity→AEX→HIC→MMC | Log 1 | 0 | 0.5 | No control |
| | reduction | | | |
| Affinity→AEX→HIC | Log 1 | 0 | 1 | No control |
| | reduction | | | |
| Affinity→HIC→ AEX→MMC | Log 1 | 0 | 1 | Up to 5% |
| | reduction | | | reduction |
| Affinity→HIC→MMC→AEX | Log 1 | 0 | 0.5 | Up to 7% |
| | reduction | | | reduction |

1. A method of purifying an Fc-fusion protein from a composition comprising the Fc-fusion protein and one or more contaminants, the method comprising chromatography steps in the order of:
a. Affinity chromatography,
b. Hydrophobic Interaction chromatography,
c. Mixed mode chromatography, and
d. Anion-exchange chromatography.

2. The method as claimed in claim 1, wherein the mixed-mode chromatography follows the hydrophobic interaction chromatography and precedes the anion exchange chromatography.

3. The method as claimed in claim 1, wherein the anion exchange chromatography is the final chromatographic step for the purification of the said Fc-fusion protein.

4. The method as claimed in claim 1, wherein the hydrophobic interaction chromatography step is placed before the anion exchange chromatography step in order to reduce more than 90% of high-molecular weight aggregates and for complete removal of non-covalent dimers.

5. The method as claimed in claim 1, wherein the mixed mode chromatography step is placed after the hydrophobic interaction chromatography step and before the anion exchange chromatography step in order to further reduce the level of high molecular weight aggregates and also to eliminate the need of additional filtration and concentration steps, which in turn prevents the loss of the Fc-fusion protein.

6. The method as claimed in claim 1, wherein the anion exchange chromatography is used as the final chromatography step in order to control the level of sialylated isoforms in the final composition within a target range.

7. The method as claimed in claim 1, wherein the anion exchange chromatography is performed in bind-elute mode.

8. The method as claimed in claim 1, wherein the hydrophobic interaction chromatography and the mixed-mode chromatography are performed in flow-through mode.

9. The method as claimed in claim 1, wherein the Fc-fusion protein is CTLA4-Ig fusion protein.

10. The method as claimed in claim 1, wherein the Fc-fusion protein is abatacept.
